(19) United States
(12) Patent Application Publication
(10) Pub. No.: US 2014/0213801 A1
(43) Pub. Date: Jul. 31, 2014

(54) MULTILAYER CATALYST HAVING VANADIUM ANTIMONATE IN AT LEAST ONE CATALYST LAYER FOR PREPARING CARBOXYLIC ACIDS AND/OR CARBOXYLIC ANHYDRIDES AND PROCESS FOR PREPARING PHTHALIC ANHYDRIDE HAVING A LOW HOT SPOT TEMPERATURE

(71) Applicants: Stefan Altwasser, Wachenheim (DE); Jürgen Zühlke, Speyer (DE); Frank Rosowski, Mannheim (DE); Cornelia Katharina Dobner, Ludwigshafen (DE)

(72) Inventors: Stefan Altwasser, Wachenheim (DE); Jürgen Zühlke, Speyer (DE); Frank Rosowski, Mannheim (DE); Cornelia Katharina Dobner, Ludwigshafen (DE)

(73) Assignee: BASF SE, Ludwigshafen (DE)

(21) Appl. No.: 14/242,979

(22) Filed: Apr. 2, 2014

Related U.S. Application Data
(62) Division of application No. 12/950,140, filed on Nov. 19, 2010.
(60) Provisional application No. 61/262,938, filed on Nov. 20, 2009.

(51) Int. Cl. C07D 307/89

(52) U.S. Cl. CPC C07D 307/89
USPC 549/248

(57) ABSTRACT

The present invention relates to a catalyst system for preparing carboxylic acids and/or carboxylic anhydrides, which system comprises a plurality of superposed catalyst layers arranged in a reaction tube, where vanadium antimonate is introduced into the active material in at least one of the catalyst layers. The present invention further relates to a process for gas-phase oxidation, in which a gaseous stream comprising at least one hydrocarbon and molecular oxygen is passed through a plurality of catalyst layers and the maximum hot spot temperature is below 425° C.

### CROSS-REFERENCE TO RELATED APPLICATIONS

This application is a divisional of application Ser. No. 12/950,140 filed on Nov. 19, 2010, the contents of which are incorporated herein by reference in its entirety, and which claims the benefit of U.S. Provisional Application Ser. No. 61/262,938 filed on Nov. 20, 2009, the contents of which are incorporated herein by reference in its entirety.

### FIELD OF THE INVENTION

The present invention relates to a catalyst system for preparing carboxylic acids and/or carboxylic anhydrides, which system comprises a plurality of superposed catalyst layers arranged in a reaction tube, where vanadium antimonate is introduced into the active catalyst material in at least one of the catalyst layers. The present invention further relates to a process for gas-phase oxidation, in which a gaseous stream comprising at least one hydrocarbon and molecular oxygen is passed through a plurality of catalyst layers and the maximum hot spot temperature is below 425° C.

### BACKGROUND OF THE INVENTION

Many carboxylic acids and/or carboxylic anhydrides are prepared industrially by catalytic gas-phase oxidation of hydrocarbons such as benzene, xylenes, naphthalene, toluene or durene in fixed-bed reactors. It is in this way possible to obtain, for example, benzoic acid, maleic anhydride, phthalic anhydride, isophthalic acid, terephthalic acid or pyromellitic anhydride. In general, a mixture of an oxygen-comprising gas and the starting material to be oxidized is passed through tubes in which a bed of a catalyst is present. To regulate the temperature, the tubes are surrounded by a heat transfer medium, for example a salt melt.

The catalysts used in the process of the invention are generally coated catalysts in which the catalytically active material has been applied in the form of a shell to an inert support. The shell thickness of the catalytically active material is generally from 0.02 to 0.25 mm, preferably from 0.05 to 0.15 mm. The proportion of active composition in the catalyst is usually from 5 to 25% by weight, mostly from 7 to 15% by weight. In general, the catalysts have a shell of active material having an essentially homogeneous chemical composition. Furthermore, two or more different shells of active material can also be applied in succession to a support. This is then referred to as a two-shell or multishell catalyst (see, for example, DE 19839001 A1).

As inert support material, it is possible to use virtually all support materials of the prior art which are advantageously employed in the production of coated catalysts for the oxidation of aromatic hydrocarbons to aldehydes, carboxylic acids and/or carboxylic anhydrides, as described, for example, in WO 2004/103561. Preference is given to using steatite in the form of spheres having a diameter of from 3 to 6 mm or of rings having an external diameter of from 5 to 9 mm, a length of from 4 to 7 mm and an internal diameter of from 3 to 7 mm.

Titanium dioxide is usually used in the anatase form for the catalytically active composition. The titanium dioxide preferably has a BET surface area of from 15 to 60 m²/g, in particular from 15 to 45 m²/g, particularly preferably from 13 to 28 m²/g. The titanium dioxide used can comprise a single titanium dioxide or a mixture of titanium dioxides. In the latter case, the value of the BET surface area is the weight average of the contributions of the individual titanium dioxides. The titanium dioxide used advantageously comprises, for example, a mixture of a TiO₂ having a BET surface area of from 5 to 15 m²/g and a TiO₂ having a BET surface area of from 15 to 50 m²/g.

A suitable vanadium source is, in particular, vanadium pentoxide or ammonium metavanadate. Suitable antimony sources are various antimony oxides. Possible phosphorus sources are, in particular, phosphoric acid, phosphorous acid, hypophosphorous acid, ammonium phosphate or phosphoric esters and especially ammonium dihydrogenphosphate. Possible sources of cesium are the oxide or hydroxide or the salts which can be thermally converted into the oxide, for example carboxylates, in particular the acetate, malonate or oxalate, carbonate, hydrogencarbonate, sulfate or nitrate.

Apart from the optional additions of cesium and phosphorus, small amounts of many other oxidic compounds which act as promoters to influence the activity and selectivity of the catalyst, for example by decreasing or increasing its activity, can be comprised in the catalytically active composition. As promoters, mention may be made by way of example of the alkali metals, in particular the abovementioned cesium and also lithium, potassium and rubidium, which are usually used in the form of their oxides or hydroxides, thallium(I) oxide, aluminum oxide, zirconium oxide, iron oxide, nickel oxide, cobalt oxide, manganese oxide, tin oxide, silver oxide, copper oxide, chromium oxide, molybdenum oxide, tungsten oxide, iridium oxide, tantalum oxide, niobium oxide, arsenic oxide, antimony tetroxide, antimony pentoxide and cerium oxide.

Among the promoters mentioned, preferred additives are the oxides of niobium and tungsten in amounts of from 0.01 to 0.50% by weight, based on the catalytically active composition.

The application of the individual shells of the coated catalyst can be carried out by any methods known per se, e.g. by spraying of solutions or suspensions onto the support in a coating drum or coating with a solution or suspension in a fluidized bed, as described, for example, in WO 2005/030388, DE 4006935 A1, DE 19824532 A1, EP 0966324 B1. Organic binders, preferably copolymers, advantageously in the form of an aqueous dispersion, of acrylic acid-maleic acid, vinyl acetate-vinyl laurate, vinyl acetate-acrylate, styrene-acrylate and vinyl acetate-ethylene, are generally added to the suspensions used. The binders are commercially available as aqueous dispersions having a solids content of, for example, from 35 to 65% by weight. The amount of such binder dispersions used is generally from 2 to 45% by weight, preferably from 5 to 35% by weight, particularly preferably from 7 to 20% by weight, based on the weight of the suspension.

The support is fluidized in, for example, a fluidized-bed or moving-bed apparatus in an ascending gas stream, in particular air. The apparatuses usually comprise a conical or spherical vessel into which the fluidizing gas is introduced from below or from the top via an immersed tube. The suspension is sprayed via nozzles from the top, from the side or from below into the fluidized bed. The use of a riser tube arranged centrally within or concentrically around the immersed tube is advantageous. A higher gas velocity which transports the support particles upward prevails within the riser tube. In the outer ring, the gas velocity is only a little above the loosening velocity. In this way, the particles are moved circularly and vertically. A suitable fluidized-bed apparatus is described, for example, in DE-A 4006935.

When coating the catalyst support with the catalytically active composition, coating temperatures of from 20 to 500° C. are generally employed, with coating being able to be carried out under atmospheric pressure or under reduced pressure. In general, coating is carried out at from 0° C. to 200° C., preferably from 20 to 150° C., in particular from 60 to 120° C.

As a result of the thermal treatment of the resulting precatalyst at temperatures of from >200 to 500° C., the binder is driven off from the applied layer by thermal decomposition and/or combustion. The thermal treatment is preferably carried out in situ in the gas-phase oxidation reactor.

The Japanese published specification No. 180430/82 discloses two-layer catalysts comprising titanium dioxide and vanadium antimonate as catalytically active components for the oxidation of o-xylene to phthalic anhydride. However, the possible o-xylene loadings and the space velocities are limited in the case of these catalysts.

The hot spot temperatures in, for example, the oxidation of o-xylene to phthalic anhydride (PA) at loadings in the range from 80 to 100 g of o-xylene/standard m³ are usually above 440° C. High hot spot temperatures reflect an excessive increase in the total oxidation of o-xylene to CO, CO₂ and water and are associated with increased damage to the catalyst. The lowest possible hot spot temperatures are therefore desirable.

### BRIEF SUMMARY OF THE INVENTION

It was an object of the present invention to provide an improved catalyst for preparing carboxylic acids and/or carboxylic anhydrides, in particular an improved catalyst for the partial oxidation of o-xylene to PA for o-xylene loadings of at least 80 g/standard m³.

The object is achieved by a multilayer catalyst for preparing carboxylic acids and/or carboxylic anhydrides which has at least 3 layers and in the production of which a vanadium antimonate is added to at least one catalyst layer. The hot spot temperature of such a catalyst is overall significantly lower than in the case of a comparable catalyst which was produced without addition of vanadium antimonate, and the carboxylic acid or carboxylic anhydride yields are significantly higher.

### DETAILED DESCRIPTION OF THE PREFERRED EMBODIMENTS

The vanadium antimonate introduced into at least one layer in the active material can be prepared by reaction of any vanadium and antimony compounds. Direct reaction of the oxides to give a mixed oxide or vanadium antimonate is preferred. The vanadium antimonate can have various molar ratios of V/Sb and can also, if appropriate, comprise further vanadium or antimony compounds and can be used in admixture with further vanadium or antimony compounds. The preparation of the vanadium antimonate can, for example, involve reaction of the oxides in aqueous solution or the use of hydrogen peroxide. In the latter case, for example, vanadium pentoxide can be dissolved in an aqueous hydrogen peroxide solution and subsequently reacted with antimony trioxide to form vanadium antimonate.

In a preferred embodiment, the catalysts of the invention comprise three, four or five layers and can, for example to avoid high hot spot temperatures, also be used in combination with suitable upstream and/or downstream beds or together with intermediate layers, with the upstream and/or downstream beds and the intermediate layers generally being able to comprise catalytically inactive or less active material.

The invention further provides a process for producing a multilayer catalyst for preparing carboxylic acids and/or carboxylic anhydrides which has at least 3 layers, wherein a vanadium antimonate is added to at least one catalyst layer.

The invention further provides a process for the gas-phase oxidation of hydrocarbons over a multilayer catalyst which has at least 3 layers and in the production of which a vanadium antimonate is added to at least one catalyst layer. The process of the invention is preferred for the gas-phase oxidation of aromatic C6-C10-hydrocarbons such as benzene, xylenes, toluene, naphthalene or durene (1,2,4,5-tetramethyl-benzene) to carboxylic acids and/or carboxylic anhydrides such as maleic anhydride, phthalic anhydride, benzoic acid and/or pyromellitic dianhydride. The process is particularly suitable for the preparation of phthalic anhydride from o-xylene and/or naphthalene. Gas-phase reactions for preparing phthalic anhydride are generally known and are described, for example, in WO 2004/103561.

In a preferred embodiment of the process of the invention, the hot spot temperature is not above 425° C. in any of the catalyst layers.

The invention further provides for the use of a multilayer catalyst which has at least 3 layers and in the production of which a vanadium antimonate is added to at least one catalyst layer for preparing carboxylic acids and/or carboxylic anhydrides.

### EXAMPLES

### Example 1

### According to the Invention

### Catalyst Layer 1 (CL1) (Vanadium Antimonate as V and Sb Source):

### Preparation of the Vanadium Antimonate:

6 l of demineralized water were placed in a thermostated double-walled glass vessel. 2855.1 g of vanadium pentoxide and 1827.5 g of antimony trioxide were suspended therein. Further rinsing-in with a further liter of demineralized water was subsequently carried out, the suspension was heated to 100° C. while stirring and after 100° C. had been reached was stirred at this temperature for 16 hours. The suspension was subsequently cooled to 80° C. and dried by spray drying. The inlet temperature was 340° C., and the outlet temperature was 110° C. The spray-dried power obtained in this way had a vanadium content of 32% by weight and an antimony content of 30% by weight. The vanadium antimonate prepared in this way had a vanadium oxidation state of 4.24 and a BET surface area of 95 m²/g.

### Preparation of the Suspension and Coating:

4.44 g of cesium carbonate, 413.7 g of titanium dioxide (Fuji TA 100CT type, anatase, BET surface area: 27 m²/g), 222.1 g of titanium dioxide (Fuji TA 100 type, anatase, BET surface area: 7 m²/g) and 91.6 g of vanadium antimonate were suspended in 1869 g of demineralized water and stirred for 18 hours to achieve a homogeneous distribution. 78.4 g of organic binders comprising a copolymer of vinyl acetate and vinyl laurate were added in the form of a 50 wt.-% aqueous dispersion to this suspension. In a fluidized-bed apparatus, 768 g of this suspension were sprayed onto 2 kg of steatite (magnesium silicate) in the form of rings having dimensions of 7 mm×7 mm×4 mm and dried.

After calcination of the catalyst at 450° C. for one hour, the amount of active material applied to the steatite ring was 8.4%. The analyzed contents of the active material were 7.1% of V₂O₅, 4.5% of Sb2O₃, 0.50% of Cs, balance TiO₂.

In contrast to CL1, vanadium pentoxide and antimony trioxide were used instead of vanadium antimonate as V and Sb source for making up the suspension in the production of CL2, CL3, CL4 and CL5.

Catalyst layer 2 (CL2) (vanadium pentoxide and antimony trioxide as V and Sb source): Production analogous to CL1 with variation of the composition of the suspension. After calcination of the catalyst at 450° C. for one hour, the amount of active material applied to the steatite rings was 9.1%. The analyzed contents of the active material were 7.1% of V₂O₅, 1.8% of Sb₂O₃, 0.38% of Cs, balance TiO₂ having an average BET surface area of 16 m²/g.

Catalyst layer 3 (CL3) (vanadium pentoxide and antimony trioxide as V and Sb source): Production analogous to CL1 with variation of the composition of the suspension. After calcination of the catalyst at 450° C. for one hour, the amount of active material applied to the steatite rings was 8.5%. The analyzed contents of the active material were 7.95% of V2O₅, 2.7% of Sb₂O₃, 0.31% of Cs, balance TiO₂ having an average BET surface area of 18 m²/g.

Catalyst layer 4 (CL4) (vanadium pentoxide and antimony trioxide as V and Sb source): Production analogous to CL1 with variation of the composition of the suspension. After calcination of the catalyst at 450° C. for one hour, the amount of active material applied to the steatite rings was 8.5%. The analyzed contents of the active material were 7.1% of V₂O₅, 2.4% of Sb₂O₃, 0.10% of Cs, balance TiO₂ having an average BET surface area of 17 m²/g.

### Catalyst Layer 5 (CL5):

Production analogous to CL1 with variation of the composition of the suspension. After calcination of the catalyst at 450° C. for one hour, the amount of active material applied to the steatite rings was 9.1%. The analyzed contents of the active material were 20% of V₂O₅, 0.38% of P, balance TiO₂ having an average BET surface area of 23 m²/g.

Oxidation of o-xylene to phthalic anhydride:

The catalytic oxidation of o-xylene to phthalic anhydride was carried out in a tube reactor which was cooled by means of a salt bath and had an internal diameter of the tubes of 25 mm. From the reactor inlet to the reactor outlet, 80 cm of CL1, 60 cm of CL2, 70 cm of CL3, 50 cm of CL4 and 60 cm of CL5 were introduced into a 3.5 m long iron tube having an internal diameter of 25 mm. The iron tube was surrounded by a salt melt to regulate the temperature, and a thermocouple tube having an external diameter of 4 mm and an installed withdrawable thermocouple served for measuring the catalyst temperature.

4.0 standard m³/h of air having loadings of 99.2 wt.-% o-xylene of from 30 to 100 g/standard m³ were passed through the tube from the top downward. At 80 g of o-xylene/standard m³, the results summarized in table 1 were obtained (“PA yield” is the amount of phthalic anhydride obtained in percent by weight, based on 100% pure o-xylene).

### Example 2

### Not According to the Invention

From the reactor inlet to the reactor outlet, 130 cm of CL2, 70 cm of CL3, 60 cm of CL4, 60 cm of CL5 were introduced into a 3.5 m long iron tube having an internal diameter of 25 mm. In contrast to Example 1, vanadium antimonate was not added to any of the catalyst layers.

TABLE 1

| | Example 1 | Example 2 (not |
|---|---|---|
| | (according to | according to |
| Pilot tube results | the invention) | the invention) |
| Amount of air [standard m³/h] | 4.0 | 4.0 |
| Loading [g/standard m³] | 80 | 80 |
| Period of operation [days] | 29 | 37 |
| Salt bath temperature [° C.] | 349 | 359 |
| Hot spot temperature [° C.] | 421 | 450 |
| PA yield [% by weight] | 114.7 | 113.5 |

In both examples, the content of xylene and phthalide in the reactor outlet gas was below 0.10 or below 0.15% by weight. The PA yield in Example 1 is significantly higher than that in Example 2, and the hot spot temperature in Example 1 is significantly lower than in Example 2.

### Example 3

### According to the Invention

Catalyst layer 6 (CL6) (vanadium pentoxide and antimony trioxide as V and Sb source): Production analogous to CL1 with variation of the composition of the suspension. After calcination of the catalyst at 450° C. for one hour, the amount of active material applied to the steatite rings was 8.5%. The analyzed contents of the active material were 11.0% of V2O₅, 2.4% of Sb₂O₃, 0.22% of Cs, balance TiO₂ having an average BET surface area of 21 m²/g.

Oxidation of o-xylene to Phthalic Anhydride:

From the reactor inlet to the reactor outlet, 80 cm of CL1, 60 cm of CL2, 70 cm of CL3, 50 cm of CL6 and 60 cm of CL5 were installed. 4.0 standard m³/h of air having loadings of 99.2 wt.-% o-xylene of from 30 to 100 g/standard m³ were passed through the tube from the top downward. At 80 and 100 g of o-xylene/standard m³, the results summarized in table 2 were obtained (“PA yield” is the amount of phthalic anhydride obtained in percent by weight, based on 100% pure o-xylene).

TABLE 2

| | Example 3 | Example 3 |
|---|---|---|
| | (according to | (according to |
| Pilot tube results | the invention) | the invention) |
| Amount of air [standard m³/h] | 4.0 | 4.0 |
| Loading [g/standard m³] | 80 | 100 |
| Period of operation [days] | 61 | 138 |
| Salt bath temperature [° C.] | 350.5 | 347.0 |
| Hot spot temperature [° C.] | 406 | 414 |
| PA yield [% by weight] | 114.6 | 114.6 |

### Example 4

### According to the Invention

Catalyst layer 7 (CL7) (Vanadium Antimonate as V and Sb Source):

The vanadium antimonate was prepared by a method analogous to example 1 with variation of the V/Sb ratio. The spray-dried powder obtained in this way had a vanadium content of 28.5% by weight and an antimony content of 36% by weight.

### Preparation of the Suspension and Coating:

See example 1 with variation of the composition of the suspension using the vanadium antimonate from example 4.

After calcination of the catalyst at 450° C. for one hour, the amount of active material applied to the steatite rings was 8.3%. The analyzed contents of the active material were 7.1% of V₂O₅, 6.0% of Sb₂O₃, 0.50% of Cs, balance TiO₂ having an average BET surface area of 20 m²/g.

Oxidation of o-xylene to Phthalic Anhydride:

From the reactor inlet to the reactor outlet, 80 cm of CL7, 60 cm of CL2, 70 cm of CL3, 50 cm of CL6 and 60 cm of CL5 were installed. 4.0 standard m³/h of air having loadings of 99.2 wt.-% o-xylene of from 30 to 100 g/standard m³ were passed through the tube from the top downward. This gave the results summarized in table 3 (“PA yield” is the amount of phthalic anhydride obtained in percent by weight, based on 100% pure o-xylene).

### Example 5

### According to the Invention

Catalyst layer 8 (CL8) (vanadium antimonate as V and Sb source): The vanadium antimonate was prepared by a method analogous to example 1 with variation of the V/Sb ratio. The spray-dried powder obtained in this way had a vanadium content of 35% by weight and an antimony content of 25.5% by weight.

### Preparation of the Suspension and Coating:

See example 1 with variation of the composition of the suspension using the vanadium antimonate from example 5.

After calcination of the catalyst at 450° C. for one hour, the amount of active material applied to the steatite rings was 8.3%. The analyzed contents of the active material were 7.1% of V₂O₅, 3.5% of Sb₂O₃, 0.55% of Cs, balance TiO₂ having an average BET surface area of 20 m²/g.

Oxidation of o-xylene to Phthalic Anhydride:

From the reactor inlet to the reactor outlet, 80 cm of CL8, 60 cm of CL2, 70 cm of CL3, 50 cm of CL6 and 60 cm of CL5 were installed. 4.0 standard m³/h of air having loadings of 99.2 wt.-% o-xylene of from 30 to 100 g/standard m³ were passed through the tube from the top downward. This gave the results summarized in table 3 (“PA yield” is the amount of phthalic anhydride obtained in percent by weight, based on 100% pure o-xylene).

TABLE 3

| | Example 4 | Example 5 |
|---|---|---|
| | (according to | (according to |
| Pilot tube results | the invention) | the invention) |
| Amount of air [standard m³/h] | 4.0 | 4.0 |
| Loading [g/standard m³] | 100 | 100 |
| Period of operation [days] | 27 | 78 |
| Salt bath temperature [° C.] | 352.5 | 344.0 |
| Hot spot temperature [° C.] | 407 | 423 |
| PA yield [% by weight] | 113.9 | 114.1 |

### Example 6

### Not According to the Invention

Catalyst layer 9 (CL9) (vanadium pentoxide and antimony trioxide as V and Sb source): Production analogous to CL1 with variation of the composition of the suspension. After calcination of the catalyst at 450° C. for one hour, the amount of active material applied to the steatite rings was 8.5%. The analyzed contents of the active material were 7.1% of V₂O₅, 6.0% of Sb₂O₃, 0.38% of Cs, balance TiO₂ having an average BET surface area of 20 m²/g.

Oxidation of o-xylene to Phthalic Anhydride:

From the reactor inlet to the reactor outet, 80 cm of CL9, 60 cm of CL2, 60 cm of CL3, 60 cm of CL6 and 60 cm of CL5 were installed. 4.0 standard m³/h of air having loadings of 99.2 wt.-% o-xylene of from 30 to 100 g/standard m³ were passed through the tube from the top downward. This gave the results summarized in table 4 (“PA yield” is the amount of phthalic anhydride obtained in percent by weight, based on 100% pure o-xylene).

TABLE 4

| | Example 6 (not |
|---|---|
| | according to |
| Pilot tube results | the invention) |
| Amount of air [standard m³/h] | 4.0 |
| Loading [g/standard m³] | 75 |
| Period of operation [days] | 29 |
| Salt bath temperature [° C.] | 361 |
| Hot spot temperature [° C.] | 448 |
| PA yield [% by weight] | 112.4 |

1. A process comprising oxidizing o-xylene to phthalic anhydride over a multilayer catalyst, wherein the o-xylene loading is at least 80 g/standard m³, wherein the multilayer catalyst comprises at least three layers, wherein a vanadium antimonate is added to at least one catalyst layer in the production of the catalyst, and wherein a hot spot temperature is not above 425° C. in any of the catalyst layers.

2. The process according to claim 1, wherein the vanadium antimonate is added to the first layer of the multilayer catalyst in the flow direction.

3. The process according to claim 1, wherein the proportion of an active composition in the catalyst is from 5 to 25% by weight.

4. The process according to claim 1, wherein the multilayer catalyst comprises an inert support and a shell comprising a catalytically active material and wherein the shell is applied to the inert support.

5. The process according to claim 4, wherein the inert support is comprised of steatite spheres having a diameter of from 3 to 6 mm.

6. The process according to claim 4, wherein the inert support is comprised of steatite rings having an external diameter of from 5 to 9 mm.
